(19) United States
(12) Patent Application Publication
(10) Pub. No.: US 2025/0073406 A1
(43) Pub. Date: Mar. 6, 2025

(54) CENTRAL MONITORING SYSTEM AND METHOD USING ARTIFICIAL VENTILATORS

(71) Applicant: THE CATHOLIC UNIVERSITY OF KOREA INDUSTRY-ACADEMIC COOPERATION FOUNDATION, Seoul (KR)

(72) Inventors: Jae Hoon SONG, Seoul (KR); Seong Hun CHANG, Goyang-si, Gyeonggi-do (KR); Hyun Hun KIM, Goyang-si, Gyeonggi-do (KR); Gyung Tae KIM, Goyang-si, Gyeonggi-do (KR); Dong Wook SHIN, Seoul (KR)

(21) Appl. No.: 18/723,056

(22) PCT Filed: Sep. 16, 2022
(86) PCT No.: PCT/KR2022/013857

§ 371 Date: Jun. 21, 2024

(30) Foreign Application Priority Data
Dec. 21, 2021 (KR) 10-2021-0184255

(51) Int. Cl. A61M 16/00

(52) U.S. Cl. CPC A61M 16/0051; A61M 2205/18; A61M 2205/502; A61M 2205/84

(57) ABSTRACT

A central monitoring system using artificial ventilators, comprises: a plurality of artificial ventilators positioned inside a hospital room in a ward and connected to the respiratory tracts of a plurality of patients recovering inside the hospital room, so as to supply oxygen to the plurality of patients; an image-capturing unit positioned inside the hospital room so as to capture the plurality of patients in real time; a communication unit for receiving a plurality of pieces of patient condition information transmitted from the plurality of artificial ventilators and patient-capturing information transmitted from the image-capturing unit; and a communication connection unit for connecting the plurality of artificial ventilators and the communication unit, and transmitting, to the communication unit, the plurality of pieces of patient condition information transmitted from the plurality of artificial ventilators.

### TECHNICAL FIELD

The present invention relates to a central monitoring system and method using artificial ventilators and, more specifically, to a central monitoring system and method using artificial ventilators for monitoring the conditions of a plurality of patients inside a hospital room in real time through a display unit positioned at a station outside the hospital room.

### BACKGROUND ART

In operating rooms equipped in medical facilities such as hospitals, surgical staff perform surgeries by referring to medical information provided by measuring the patient's condition, and accordingly, various medical equipment is equipped in the operating room.

The medical equipment as mentioned above includes bio-signal measuring devices, imaging diagnostic devices, respiratory therapy devices, and biochemical analyzers. The bio-signal measuring devices refer to electrocardiogram measuring devices, blood pressure measuring devices, oxygen saturation measuring devices, and body temperature measuring devices, and respiratory therapy devices refer to artificial ventilators and high-flow respiratory therapy devices, and imaging diagnostic devices refer to ultrasound diagnostic devices, electrical impedance tomography devices, etc. In addition, biochemical analyzers refer to blood analyzers, urine analyzers, and body fluid component analyzers.

Each of these medical devices may be equipped with power cables, cords, and tubes, which may make the operating room complicated. In addition, the medical equipment itself is equipped with display panels and control panels, etc., so that the surgeon or nurses are able to move to the location of each medical equipment during surgery and monitor the patient's condition.

In this complex operating room environment, the floor and cables may easily be contaminated by blood and secretions generated during the surgery, and if the surgical equipment contaminated with blood or secretions is reused in another operating room, secondary infection may occur due to blood and secretions on the surgical equipment cables.

Therefore, in the case of urgent surgery, it is difficult to efficiently manage the patient's condition or a lot of manpower is required for this.

In addition, due to COVID-19, which has been spreading widely around the world since 2019, the government has ordered additional emergency treatment beds for COVID-19 patients.

Accordingly, hospitals should operate in a way that restricts or minimizes entry into each hospital room.

Currently, patient monitoring devices can be monitored through the central patient monitoring device at the station, but information related to artificial ventilators cannot be monitored at the station.

Accordingly, a central monitoring system that integrates information related to the conditions of a plurality of patients recovering inside the hospital room and enables integrated management that may be recognized at a glance from an external station is urgently needed.

(Patent Document 1) Publication of Patent Publication No. 10-2017-0001796 (Jan. 5, 2017)

### DETAILED DESCRIPTION OF THE INVENTION

### Technical Problem

An aspect of the present invention is to provide a central monitoring system and method using artificial ventilators, wherein a plurality of pieces of patient condition information transmitted from a plurality of artificial ventilators may be monitored in real time through a single display unit positioned outside a hospital room, and wherein an emergency patient is determined based on a plurality of pieces of patient condition information, and then an alarm sound is generated while transmitting an emergency signal and patient-capturing information of an emergency patient to a user terminal owned by a doctor in charge.

The aspect of the present invention is not limited to that mentioned above, and other aspects not mentioned will be clearly understood by those skilled in the art from the description below.

### Technical Solution

The present invention provides a central monitoring system using artificial ventilators, the system including: a plurality of artificial ventilators positioned inside a hospital room in a ward and connected to the respiratory tracts of a plurality of patients recovering inside the hospital room, so as to supply oxygen to the plurality of patients; an image-capturing unit positioned inside the hospital room so as to capture the plurality of patients in real time; a communication unit for receiving a plurality of pieces of patient condition information transmitted from the plurality of artificial ventilators and patient-capturing information transmitted from the image-capturing unit; a communication connection unit for connecting the plurality of artificial ventilators and the communication unit, and transmitting, to the communication unit, the plurality of pieces of patient condition information transmitted from the plurality of artificial ventilators; a database unit in which the plurality of pieces of patient condition information and the patient-capturing information transmitted from the communication unit are stored and a plurality of pieces of preset patient condition information are pre-stored; a display unit for displaying, on a screen, the plurality of pieces of patient condition information transmitted from the database unit; and a control unit, which compares the plurality of pieces of patient condition information and the plurality of pieces of preset patient condition information so as to determine an emergency patient, and generates, if there is an emergency patient, an alarm sound and transmits patient-capturing information together with an emergency signal to a user terminal possessed by a doctor in charge.

In an embodiment of the present invention. the patient condition information may include at least one of one of air and inhalation oxygen concentrations (FiO₂), a patient's intraairway pressure, air carbon dioxide (CO₂) concentration, an air flow rate, an air velocity, a patient's exhalation carbon dioxide (CO₂) concentration, a patient's heart rate, and an oxygen saturation, and the plurality of pieces of patient condition information may include at least one of one of air and inhalation oxygen concentrations (FiO₂), a patient's intraairway pressure, air carbon dioxide (CO₂) concentration, an air flow rate, an air velocity, a patient's exhalation carbon dioxide (CO₂) concentration, a patient's heart rate, and an oxygen saturation when the plurality of patients are in a normal state.

In an embodiment of the present invention, the plurality of pieces of patient condition information may be information for checking the conditions of the plurality of patients when each of the plurality of patients is in a normal state, and as a result of comparing the plurality of pieces of patient condition information with the plurality of pieces of preset patient condition information, if any one of the plurality of pieces of patient condition information deviates from any one of the plurality of pieces of preset patient condition information, the control unit may determine a patient for which the one of the patient condition information is measured as an emergency patient, generate an alarm sound, and transmit the patient-capturing information together with an emergency signal to the user terminal possessed by the doctor in charge.

In an embodiment of the present invention, the display unit may display, by flashing in red, the patient condition information of the patient determined to be the emergency patient among the plurality of pieces of patient condition information displayed on the screen.

In an embodiment of the present invention, the plurality of patients may be a first patient, a second patient, a third patient, and a fourth patient who are recovering in four bedrooms arranged inside the hospital room, respectively, and the plurality of artificial ventilators may be: a first artificial ventilator that supplies the oxygen to the first patient and transmits first patient condition information among the plurality of pieces of patient condition information to the communication unit; a second artificial ventilator that supplies the oxygen to the second patient and transmits second patient condition information among the plurality of pieces of patient condition information to the communication unit; a third artificial ventilator that supplies the oxygen to the third patient and transmits third patient condition information among the plurality of pieces of patient condition information to the communication unit; and a fourth artificial ventilator that supplies the oxygen to the fourth patient and transmits fourth patient condition information among the plurality of pieces of patient condition information to the communication unit.

In an embodiment of the present invention, the plurality of pieces of preset patient condition information may be first preset patient condition information when the first patient is in a normal state, second preset patient condition information when the second patient is in a normal state, third preset patient condition information when the third patient is in a normal state, and fourth preset patient condition information when the fourth patient is in a normal state, the database unit may transmit the first to fourth patient condition information transmitted from the communication unit to the display unit, and the control unit may compare the first to fourth patient condition information with the first to fourth preset patient condition information pre-stored in the database unit to determine whether the emergency patient is among the first to fourth patients.

The present invention provides a central monitoring method using artificial ventilators, the central monitoring method using the central monitoring system using artificial ventilators as described above, the central monitoring method including the steps in which: (a) the plurality of artificial ventilators supplying the oxygen to the plurality of patients transmit the plurality of pieces of patient condition information to the communication unit; (b) the image-capturing unit transmits the patient-capturing information obtained by capturing the plurality of patients in real time to the communication unit; (c) the database unit stores the plurality of pieces of patient condition information and the patient-capturing information transmitted from the communication unit; (d) the display unit displays the plurality of pieces of patient condition information on a screen; and (e) the control unit notifies an emergency situation based on a result of comparing the plurality of pieces of patient condition information with the plurality of pieces of preset patient condition information.

In an embodiment of the present invention, the plurality of patients may be a first patient, a second patient, a third patient. and a fourth patient who are recovering in the four bedrooms arranged inside the hospital room, respectively, and step (a) may include the steps in which: (a1) a first artificial ventilator among the plurality of artificial ventilators, which supplies the oxygen to the first patient, transmits first patient condition information to the communication unit; (a2) a second artificial ventilator among the plurality of artificial ventilators, which supplies the oxygen to the second patient, transmits second patient condition information to the communication unit; (a3) a third artificial ventilator among the plurality of artificial ventilators, which supplies the oxygen to the third patient, transmits third patient condition information to the communication unit; and (a4) a fourth artificial ventilator among the plurality of artificial ventilators, which supplies the oxygen to the fourth patient, transmits fourth patient condition information to the communication unit.

In an embodiment of the present invention, in step (d), the display unit may display the first to fourth patient condition information on the screen which is divided into four.

In an embodiment of the present invention, step (e) may include the steps in which: (e1) the control unit receives the first to fourth patient condition information and the first to fourth preset patient condition information that were pre-stored in the database unit; (e2) the control unit compares the first to fourth patient condition information with the first to fourth preset patient condition information, respectively, to determine whether there is the emergency patient among the first to fourth patients; (e3) the control unit determines a patient for which at least one of the first to fourth patient condition information is measured as the emergency patient, if at least one of the first to fourth patient condition information deviates from at least one of the first to fourth preset patient condition information; (e4) the control unit generates the alarm sound; (e5) the control unit controls the display unit so that the at least one piece of patient condition information measured from the emergency patient among the plurality of pieces of patient condition information displayed on the display unit flashes in red; and (e6) the control unit transmits the emergency signal and the patient-capturing information to the user terminal possessed by the doctor in charge.

### Advantageous Effects

According to the present invention, a plurality of pieces of patient condition information transmitted from a plurality of artificial ventilators may be monitored in real time through a single display unit outside the hospital room, and based on a plurality of pieces of patient condition information, it is possible to determine emergency patients and generate an alarm sound while simultaneously transmitting an emergency signal and patient-capturing information of an emergency patient to a user terminal possessed by a doctor in charge, so that admission to a hospital room may be minimized, an artificial ventilator mode may be adjusted for prescription according to the condition of the emergency patient even from outside. and rapid response is possible even in the event of an emergency.

The effects of the present invention are not limited to the effects described above, and should be understood to include all effects that are inferable from the configuration of the present invention described in the detailed description or claims of the present invention.

### BRIEF DESCRIPTION OF THE DRAWINGS

FIG. 1 is a block diagram schematically showing a central monitoring system using artificial ventilators according to an embodiment of the present invention.

FIG. 2 is a block diagram showing a central monitoring system using artificial ventilators according to an embodiment of the present invention.

FIG. 3 is a flow chart showing the operation of a central monitoring system using artificial ventilators according to an embodiment of the present invention.

FIGS. 4A and 4B are each a drawing showing the actual appearance of a central monitoring system using artificial ventilators according to an embodiment of the present invention.

### BEST MODE FOR CARRYING OUT THE INVENTION

The most preferred embodiment according to the present invention includes: a plurality of artificial ventilators positioned inside a hospital room in a ward and connected to the respiratory tracts of a plurality of patients recovering inside the hospital room, so as to supply oxygen to the plurality of patients; an image-capturing unit positioned inside the hospital room so as to capture the plurality of patients in real time; a communication unit for receiving a plurality of pieces of patient condition information transmitted from the plurality of artificial ventilators and patient-capturing information transmitted from the image-capturing unit; a communication connection unit for connecting the plurality of artificial ventilators and the communication unit, and transmitting, to the communication unit. the plurality of pieces of patient condition information transmitted from the plurality of artificial ventilators; a database unit in which the plurality of pieces of patient condition information and the patient-capturing information transmitted from the communication unit are stored and a plurality of pieces of preset patient condition information are pre-stored; a display unit for displaying, on a screen, the plurality of pieces of patient condition information transmitted from the database unit; and a control unit, which compares the plurality of pieces of patient condition information and the plurality of pieces of preset patient condition information so as to determine an emergency patient, and generates, if there is an emergency patient, an alarm sound and transmits patient-capturing information together with an emergency signal to a user terminal possessed by a doctor in charge.

### Mode for Carrying Out the Invention

Hereinafter, the present invention will be described with reference to the accompanying drawings. However, the present invention may be implemented in various different forms and, therefore, is not limited to the examples described herein. In order to clearly explain the present invention in the drawings, portions unrelated to the description are omitted, and similar portions are given similar reference numerals throughout the specification.

Throughout the specification, when a portion is said to be “connected (linked, contacted, combined)” with another portion, this includes not only a case of being “directly connected” but also a case of being “indirectly connected” with another member in between. In addition, when a portion is said to “include” a certain component, this does not mean that other components are excluded, but that other components may be added, unless specifically stated to the contrary.

The terms used herein are merely used to describe specific embodiments and are not intended to limit the present invention. Singular expressions include plural expressions unless the context clearly dictates otherwise. In this specification. it should be understood terms such as “include” or “have” are to designate the presence of features, numbers, steps, operations, components, parts, or combinations thereof described in the specification, but are not to exclude in advance the possibility of the presence or addition of one or more other features, numbers, steps, operations, components, parts, or combinations thereof. Hereinafter, embodiments of the present invention will be described in detail with reference to the accompanying drawings.

### 1. Central Monitoring System 100 Using Artificial Ventilators

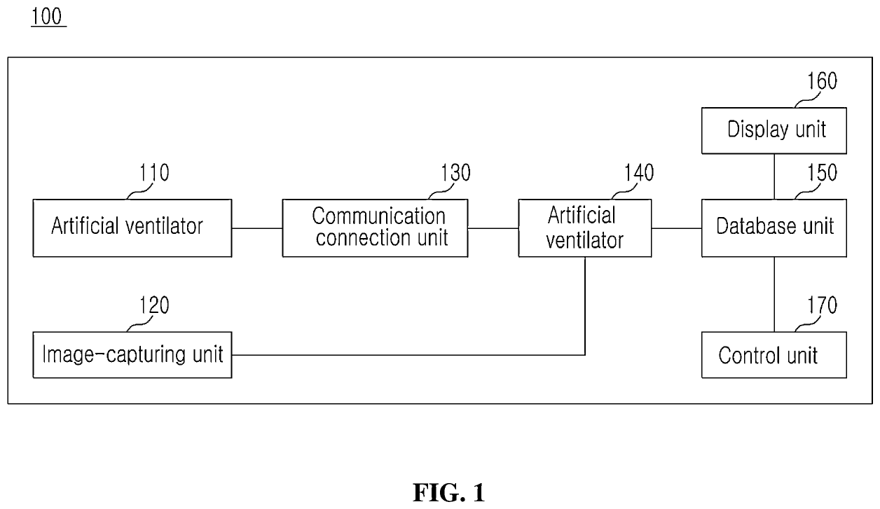

Hereinafter, a central monitoring system using artificial ventilators according to an embodiment of the present invention will be described with reference to FIGS. 1 to 4.

FIG. 1 is a block diagram schematically showing a central monitoring system using artificial ventilators according to an embodiment of the present invention.

A central monitoring system 100 using artificial ventilators according to an embodiment of the present invention includes an artificial ventilator 110, an image-capturing unit 120, a communication connection unit 130, a communication unit 140, a database unit 150, a display unit 160, and a control unit 170.

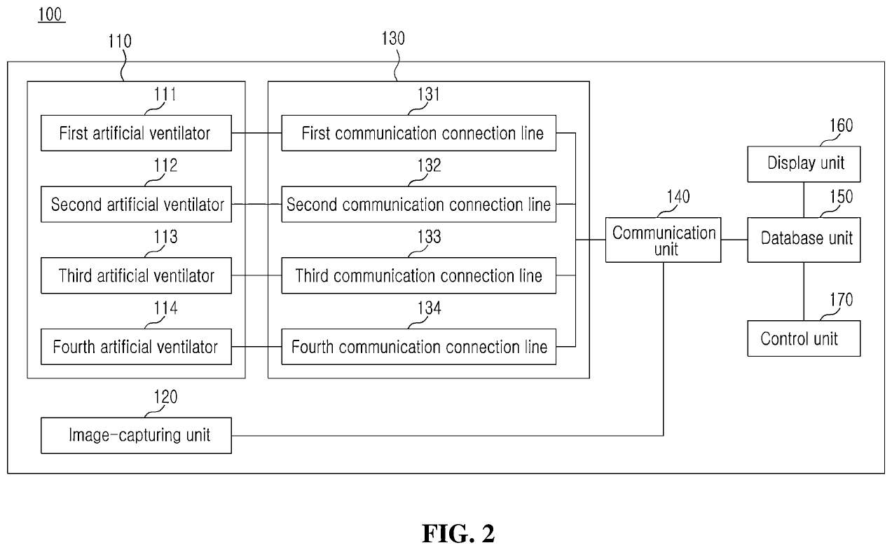

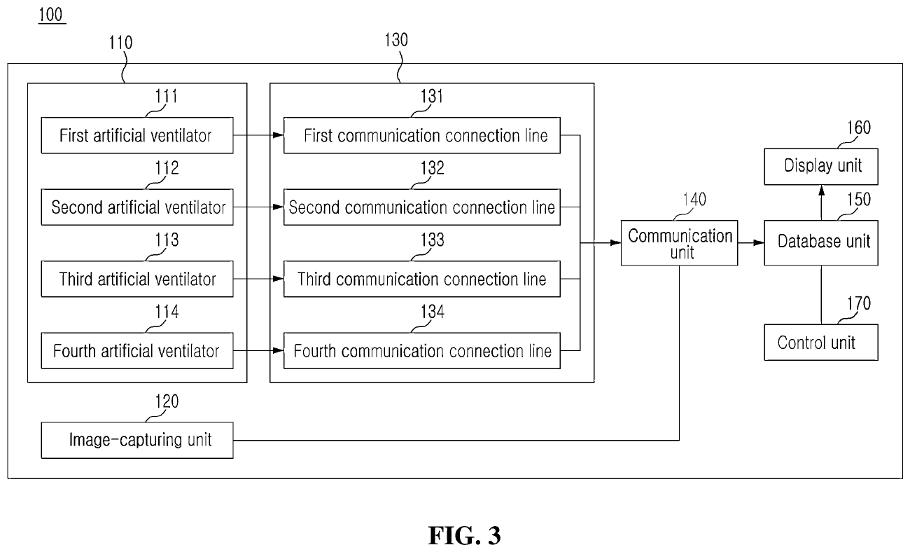

FIG. 2 is a block diagram showing a central monitoring system using artificial ventilators according to an embodiment of the present invention. FIG. 3 is a flow chart showing the operation of a central monitoring system using artificial ventilators according to an embodiment of the present invention.

Referring to FIGS. 2 and 3, an artificial ventilator 110 is positioned inside a hospital room within a ward and is connected to the respiratory tracts 111, 112, 113, 114 of a plurality of patients recovering inside the hospital room to supply oxygen to the plurality of patients.

Here, for convenience of explanation, the plurality of patients are exemplified as patients 1, 2, 3, and 4, each recovering in four bedrooms arranged inside a hospital room, but are not limited thereto.

In addition, an artificial ventilator 100 generates a plurality of pieces of patient condition information for checking the conditions of a plurality of patients.

Here, the patient condition information may include at least one of one of air and inhalation oxygen concentrations (FiO₂), a patient's airway pressure, a carbon dioxide (CO₂) concentration of the air, an air flow rate, an air velocity, a carbon dioxide (CO₂) concentration during a patient's exhalation, a patient's heart rate, and a patient's oxygen saturation.

Additionally, patient condition information may further include, in addition to the above, End-Tidal CO₂ (EtCO₂), Tidal Volume (VT), Inspiration Time (Tinsp), Frequency (F), Inspiratory Pressure (Pinsp), Spontaneous Ventilation Assistance Pressure (PASB above peep), Positive End-Expiratory Pressure (PEEP), Trigger, Minute Volume (MV), Minute Leakage (MVleak), Spontaneous Ventilation (MVspn), Low Airway Pressure (Plow), Median Airway Pressure (Pmean), Highest Airway Pressure (Pmax), Patient Lung Compliance (Ppeak), Flat Inspiratory Pressure (Pplat), Resistance (R/Resistance), Body Temperature (Temperature), Expiration Time (Te/Expiration time), Required Volume during Apnea (VTApnoea), Patient Expiratory Volume (VTe), Patient Required Volume (VTi), Residual Lung Volume (Vtrap), Patient's Spontaneous Breathing Pressure (Pmin), patient's spontaneous breathing rate (Fspn), supplied breathing rate (fmand), patient mode (Patient Mode), alarm (Alarm), and error (Error).

Specifically, the plurality of pieces of patient condition information includes at least one of one of air and inhalation oxygen concentrations (FiO₂) when a plurality of patients are in a normal state, a patient's airway pressure, a carbon dioxide (CO₂) concentration of air, an air flow rate, an air velocity, a carbon dioxide (CO₂) concentration when a patient exhales, a patient's heart rate, and an oxygen saturation.

The artificial ventilators 110 therefor include a first artificial ventilator 111, a second artificial ventilator 112, a third artificial ventilator 113, and a fourth artificial ventilator 114.

The first artificial ventilator 111 supplies oxygen to the first patient and transmits the first patient condition information among the plurality of pieces of patient condition information to the communication unit 140.

The second artificial ventilator 112 supplies oxygen to the second patient and transmits the second patient condition information among the plurality of pieces of patient condition information to the communication unit 140.

The third artificial ventilator 113 supplies oxygen to the third patient and transmits the third patient condition information among the plurality of pieces of patient condition information to the communication unit 140.

The fourth artificial ventilator 114 supplies oxygen to the fourth patient and transmits the fourth patient condition information among the plurality of pieces of patient condition information to the communication unit 140.

The first to fourth patient condition information are transmitted to the communication unit 140 through the communication connection unit 130, which is a wired LAN, or are transmitted to the communication unit 140 by wireless communication.

In addition, although the present invention has described four artificial ventilators 114 as an example, it is obvious that the number of artificial ventilators 114 may be increased and applied, such as eight, twelve, or sixteen.

The image-capturing unit 120 is positioned inside the hospital room and acquires patient-capturing information by capturing a plurality of patients in real time. Here, patient-capturing information is a video or image taken in real time of a plurality of patients living inside a hospital room.

* Specifically, the image-capturing unit 120 may be installed at the inner upper corner of the hospital room so that a plurality of patients inside the hospital room can be photographed.

Meanwhile, the image-capturing unit 120 may be installed in four units so that the first to fourth patients can be photographed 1:1, but is not limited thereto.

In addition, the image-capturing unit 120 may be, for example, a CCTV, but is not limited thereto. The image-capturing unit 120 transmits patient-capturing information captured in real time for the first to fourth patients to the communication unit 140 through the communication connection unit 130.

The communication connection unit 130 connects a plurality of artificial ventilators (111, 112, 113, 114) and the communication unit 140, and transmits a plurality of pieces of patient condition information transmitted from a plurality of artificial ventilators (111, 112, 113, 114) to the communication unit 140.

For example, the communication connection unit 130 connects the artificial ventilator 110 and the communication unit 140 by wire using a LAN cable.

Meanwhile, the present invention describes that the artificial ventilator 110 and the communication unit 140 communicate by wire using the communication connection unit 130, but the artificial ventilator 110 and the communication unit 140 may communicate wirelessly without the communication connection unit 130.

The communication connection unit 130 includes a first communication connection line 131, a second communication connection line 132, a third communication connection line 133, and a fourth communication connection line 134.

The first communication connection line 131 electrically connects the first artificial ventilator 111 and the communication unit 140, and transmits the first patient condition information transmitted from the first artificial ventilator 111 to the communication unit 140.

The second communication connection line 132 electrically connects the second artificial ventilator 112 and the communication unit 140, and transmits the second patient condition information transmitted from the second artificial ventilator 112 to the communication unit 140.

The third communication connection line 133 electrically connects the third artificial ventilator 113 and the communication unit 140, and transmits the third patient condition information transmitted from the third artificial ventilator 113 to the communication unit 140.

The fourth communication connection line 134 electrically connects the fourth artificial ventilator 114 and the communication unit 140, and transmits the fourth patient condition information transmitted from the fourth artificial ventilator 114 to the communication unit 140.

In addition, although the present invention has described four communication connection lines 131, 132, 133, 134 as an example, it is obvious that the number of channels may be increased or decreased as desired by the user, such as 8, 12, or 16.

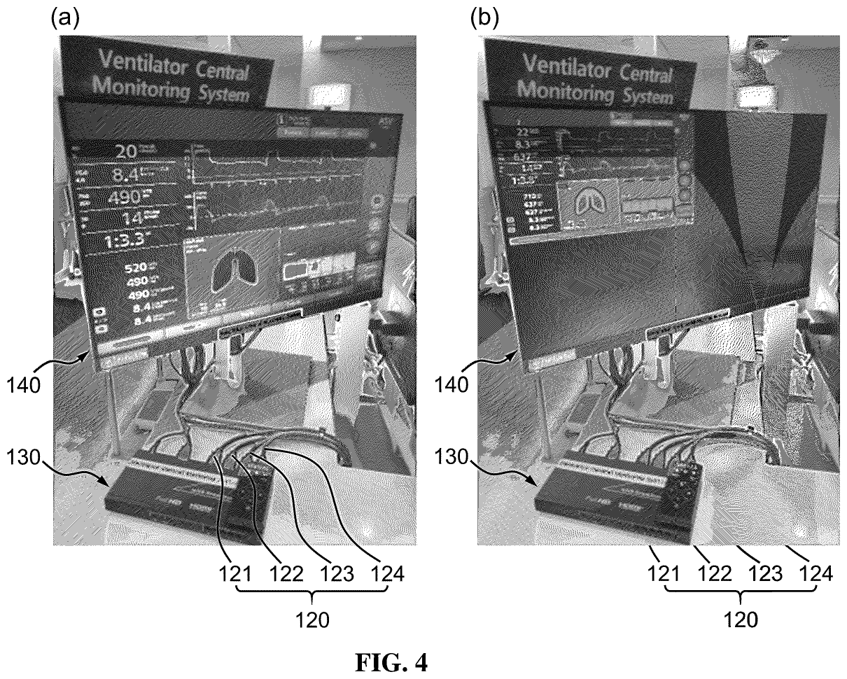

FIGS. 4A and 4B are each a drawing showing the actual appearance of a central monitoring system using artificial ventilators according to an embodiment of the present invention.

A communication unit 140 receives a plurality of pieces of patient condition information transmitted from a plurality of artificial ventilators 111, 112, 113, 114 and patient-capturing information transmitted from an image-capturing unit 120.

Referring to FIGS. 4A and 4B, the communication unit 140 acts as a hub that collects a plurality of pieces of patient condition information transmitted through a plurality of communication connection lines 131, 132, 133, 134 into a single device.

The communication unit 140 transmits a plurality of pieces of patient condition information and patient-capturing information to a database unit 150.

The database unit 150 stores a plurality of pieces of patient condition information and patient-capturing information transmitted from the communication unit 140, and a plurality of pieces of preset patient condition information is pre-stored.

Here, the plurality of pieces of preset patient condition information are information for checking the conditions of a plurality of patients when each of the plurality of patients is in a normal state.

Specifically, the plurality of pieces of preset patient condition information are first preset patient condition information when the first patient is in a normal state. second preset patient condition information when the second patient is in a normal state, third preset patient condition information when the third patient is in a normal state, and fourth preset patient condition information when the fourth patient is in a normal state.

At this time, the preset patient condition information is set to the numerical range (e.g., minimum to maximum) of each factor by collecting data on each factor (=air, an inhalation oxygen concentration, airway pressure, a carbon dioxide concentration, an air flow rate, an air velocity, a carbon dioxide concentration during a patient's exhalation, a patient's heart rate, an oxygen saturation) when the patient is in a normal state.

If the preset patient condition information is not obtained when the patient is in a normal state, the following average numerical ranges for each factor may be applied as a substitute.

In the preset patient condition information, the inhalation oxygen concentration (FiO₂) is 0.21 to 0.5, the airway pressure is the patient's airway pressure when the patient is in a normal state (for example, 5 cmH₂O to 20 cmH₂O), the carbon dioxide concentration in the air is 0.01 vol % to 1 vol %, the air flow rate is 2 to 120 L/min, the heart rate is 60 to 100 times, and the oxygen saturation is 95% to 100%.

In addition, in the present invention, patient condition information is information measured in first to fourth artificial ventilators 111, 112, 113, 114 that supply oxygen to first to fourth patients, and May include at least one of one of air and inhalation oxygen concentrations (FiO₂), a patient's intra-airway pressure, a carbon dioxide (CO₂) concentration in air, an air flow rate, an air velocity, a carbon dioxide (CO₂) concentration during a patient's exhalation, a heart rate, and an oxygen saturation of a patient.

The database unit 150 transmits the first to fourth patient condition information transmitted from the communication unit 140 to the display unit 160.

In addition, the database unit 150 transmits the first to fourth patient condition information, the first to fourth preset patient condition information, and the patient-capturing information to the control unit 170.

The display unit 160 is positioned at a station outside the hospital room, and displays a plurality of pieces of patient condition information transmitted from the database unit 150 on the screen as shown in FIGS. 4A and 4B.

In addition, the display unit 160 displays the patient condition information of a patient determined to be an emergency patient among the plurality of pieces of patient condition information displayed on the screen under the control of the control unit 170 by flashing in red.

The control unit 170 compares the first to fourth patient condition information with the first to fourth preset patient condition information stored in the database unit 150 to determine whether there is an emergency patient among the first to fourth patients.

That is, the control unit 170 compares the first patient condition information with the first preset patient condition information, compares the second patient condition information with the second preset patient condition information, compares the third patient condition information with the third preset patient condition information, and compares the fourth patient condition information with the fourth preset patient condition information.

Specifically, the control unit 170 compares a plurality of pieces of patient condition information with a plurality of pieces of preset patient condition information to determine an emergency patient, generates an alarm sound when an emergency patient occurs, and transmits patient-capturing information together with an emergency signal to a user terminal owned by a doctor in charge.

If the control unit 170 compares a plurality of pieces of patient condition information with a plurality of pieces of preset patient condition information, and if any one of the plurality of pieces of patient condition information deviates from any one of the preset patient condition information, the control unit 170 determines that the patient for which any one of the patient condition information is measured is an emergency patient, generates an alarm sound, and transmits patient-capturing information together with an emergency signal to a user terminal owned by a doctor in charge.

In addition, if it is determined that there is an emergency patient, the control unit 170 may control the operation of the first artificial ventilator 111 connected to the emergency patient (e.g., the first patient) by setting factors of the environmental state information that may convert the emergency patient (e.g., the first patient) to a normal state, or control the operation of the first artificial ventilator 111 in a mode in which the emergency patient may be converted to a normal state.

### 2. Central Monitoring Method Using Artificial Ventilators

Hereinafter, a central monitoring method using artificial ventilators according to an embodiment of the present invention will be described with reference to FIGS. 1 to 4.

A central monitoring method using artificial ventilators, which is the central monitoring method using the central monitoring system using artificial ventilators as described above, includes the steps in which: (a) the plurality of artificial ventilators 111, 112, 113, 114 supplying the oxygen to the plurality of patients transmit the plurality of pieces of patient condition information to the communication unit 140; (b) the image-capturing unit 120 transmits the patient-capturing information obtained by capturing the plurality of patients in real time to the communication unit 140; (c) the database unit 160 stores the plurality of pieces of patient condition information and the patient-capturing information transmitted from the communication unit 140; (d) the display unit 160 displays the plurality of pieces of patient condition information on a screen; and (e) the control unit 170 notifies an emergency situation based on a result of comparing the plurality of pieces of patient condition information with the plurality of pieces of preset patient condition information.

As described above, the plurality of patients are patient 1, patient 2, patient 3, and patient 4, who are recovering in four bedrooms positioned inside a hospital room, respectively.

Step (a) includes the steps in which: (a1) a first artificial ventilator 111 among the plurality of artificial ventilators 111, 112, 113, 114, which supplies the oxygen to the first patient, transmits first patient condition information to the communication unit 140; (a2) a second artificial ventilator 112 among the plurality of artificial ventilators 111, 112, 113, 114, which supplies the oxygen to the second patient, transmits second patient condition information to the communication unit 140; (a3) a third artificial ventilator 113 among the plurality of artificial ventilators 111, 112, 113, 114, which supplies the oxygen to the third patient, transmits third patient condition information to the communication unit 140; and (a4) a fourth artificial ventilator 114 among the plurality of artificial ventilators 111, 112, 113, 114, which supplies the oxygen to the fourth patient, transmits fourth patient condition information to the communication unit 140.

In steps (a1) to (a4), the first to fourth patient condition information is transmitted to the communication unit 140 through the first to fourth communication connection lines 131, 132, 133, 134.

Next, in step (b), the image-capturing unit 120 acquires patient-capturing information that has captured the first to fourth patients in real time.

However, as mentioned above, four image-capturing units 120 may be installed inside the hospital room to capture patients one-on-one.

Next, in step (c), the database unit 120 has preset patient condition information pre-stored.

The database unit 150 transmits the first to fourth patient condition information transmitted from the communication unit 140 to the display unit 160.

In addition, the database unit 150 transmits the first to fourth patient condition information, the first to fourth preset patient condition information, and the patient-capturing information to the control unit 170.

Next. in step (d), the display unit 160 displays the first to fourth patient condition information on a screen which is divided into four.

Next, step (e) includes the steps in which: (e1) the control unit receives the first to fourth patient condition information and the first to fourth preset patient condition information that were pre-stored in the database unit; (e2) the control unit compares the first to fourth patient condition information with the first to fourth preset patient condition information, respectively, to determine whether there is the emergency patient among the first to fourth patients; (e3) the control unit determines a patient for which at least one of the first to fourth patient condition information is measured as the emergency patient, if at least one of the first to fourth patient condition information deviates from at least one of the first to fourth preset patient condition information; (e4) the control unit generates the alarm sound; (e5) the control unit controls the display unit so that the at least one piece of patient condition information measured from the emergency patient among the plurality of pieces of patient condition information displayed on the display unit flashes in red; and (e6) the control unit transmits the emergency signal and the patient-capturing information to the user terminal possessed by the doctor in charge.

Here, the doctor in charge may be the attending physician or the doctor on duty for the patient who is determined to be an emergency patient.

According to the above, the present invention may monitor a plurality of pieces of patient condition information transmitted from an artificial ventilator in real time on one display unit even when confirmation by CCTV is impossible, so that when an alarm sound is generated, the patient's condition may be immediately confirmed through the display unit and then the patient may be selectively admitted to the hospital room only when necessary.

In addition, the present invention can simultaneously check the condition of a plurality of patients through one display unit, so that a quick response is possible in the event of an emergency and the time required for manpower is saved.

Moreover, the present invention allows a plurality of medical staff to check patient condition information by viewing the same screen outside the hospital room, so that even when a doctor in charge is not in the ward in the event of an emergency such as a change in the patient's condition, a quick response is possible by transmitting a video call or patient-capturing information.

The description of the present invention described above is for illustrative purposes, and those skilled in the art will understand that the present invention is easily modifiable into other specific forms without changing the technical idea or essential features of the present invention. Therefore, the examples described above should be understood in all respects as illustrative and not restrictive. For example, each component described as single may be implemented in a distributed manner, and similarly, components described as distributed may also be implemented in a combined form.

The scope of the present invention is indicated by the claims described below, and all changes or modified forms derived from the meaning and scope of the claims and their equivalent concepts should be construed as being included in the scope of the present invention.

### EXPLANATION OF REFERENCE NUMERALS

100: Central monitoring system using artificial ventilators
110: Artificial ventilator
111: First artificial ventilator
112: Second artificial ventilator
113: Third artificial ventilator

114: Fourth artificial ventilator
120: Image-capturing unit
130: Communication connection unit
131: First communication connection line
132: Second communication connection line
133: Third communication connection line
134: Fourth communication connection line
140: Communication unit
150: Database unit
160: Display unit
170: Control unit

1. A central monitoring system using artificial ventilators, the system comprising:
a plurality of artificial ventilators positioned inside a hospital room in a ward and connected to the-respiratory tracts of a plurality of patients recovering inside the hospital room, so as to supply oxygen to the plurality of patients;
an image-capturing unit positioned inside the hospital room so as to capture the plurality of patients in real time;
a communication unit for receiving a plurality of pieces of patient condition information transmitted from the plurality of artificial ventilators and patient-capturing information transmitted from the image-capturing unit;
a communication connection unit for connecting the plurality of artificial ventilators and the communication unit, and transmitting, to the communication unit, the plurality of pieces of patient condition information transmitted from the plurality of artificial ventilators;
a database unit in which the plurality of pieces of patient condition information and the patient-capturing information transmitted from the communication unit are stored and a plurality of pieces of preset patient condition information are pre-stored;
a display unit for displaying, on a screen, the plurality of pieces of patient condition information transmitted from the database unit; and
a control unit including a processor configured to compare the plurality of pieces of patient condition information and the plurality of pieces of preset patient condition information so as to determine an emergency patient, and generate, if there is an emergency patient, an alarm sound and transmits transmit patient-capturing information together with an emergency signal to a user terminal possessed by a doctor in charge.

2. The central monitoring system using artificial ventilators of claim 1, wherein the patient condition information includes at least one of one of air and inhalation oxygen concentrations (FiO₂), a patient's intraairway pressure, air carbon dioxide (CO₂) concentration, an air flow rate, an air velocity, a patient's exhalation carbon dioxide (CO₂) concentration, a patient's heart rate, and an oxygen saturation, and the plurality of pieces of patient condition information includes at least one of one of air and inhalation oxygen concentrations (FiO₂), a patient's intraairway pressure, air carbon dioxide (CO 2) concentration, an air flow rate, an air velocity, a patient's exhalation carbon dioxide (CO₂) concentration, a patient's heart rate, and an oxygen saturation when the plurality of patients are in a normal state.

3. The central monitoring system using artificial ventilators of claim 1, wherein the plurality of pieces of patient condition information are information for checking conditions of the plurality of patients when each of the plurality of patients is in a normal state, and as a result of comparing the plurality of pieces of patient condition information with the plurality of pieces of preset patient condition information, if any one of the plurality of pieces of patient condition information deviates from any one of the plurality of pieces of preset patient condition information, the control unit determines a patient for which the one of the patient condition information is measured as an emergency patient, generates an alarm sound, and transmits the patient-capturing information together with an emergency signal to the user terminal possessed by the doctor in charge.

4. The central monitoring system using artificial ventilators of claim 3, wherein the display unit displays, by flashing in red, the patient condition information of the patient determined to be the emergency patient among the plurality of pieces of patient condition information displayed on the screen.

5. The central monitoring system using artificial ventilators of claim 1, wherein the plurality of patients are-include a first patient, a second patient, a third patient, and a fourth patient who are recovering in four bedrooms arranged inside the hospital room, respectively, and the plurality of artificial ventilators include:
a first artificial ventilator that supplies the oxygen to the first patient and transmits first patient condition information among the plurality of pieces of patient condition information to the communication unit;
a second artificial ventilator that supplies the oxygen to the second patient and transmits second patient condition information among the plurality of pieces of patient condition information to the communication unit;
a third artificial ventilator that supplies the oxygen to the third patient and transmits third patient condition information among the plurality of pieces of patient condition information to the communication unit; and
a fourth artificial ventilator that supplies the oxygen to the fourth patient and transmits fourth patient condition information among the plurality of pieces of patient condition information to the communication unit.

6. The central monitoring system using artificial ventilators of claim 5, wherein the plurality of pieces of preset patient condition information include first preset patient condition information when the first patient is in a normal state, second preset patient condition information when the second patient is in a normal state, third preset patient condition information when the third patient is in a normal state, and fourth preset patient condition information when the fourth patient is in a normal state, the database unit transmits the first to fourth patient condition information transmitted from the communication unit to the display unit, and the control unit compares the first to fourth patient condition information with the first to fourth preset patient condition information pre-stored in the database unit to determine whether the emergency patient is among the first to fourth patients.

7. A central monitoring method using artificial ventilators, the central monitoring method using the central monitoring system according to claim 1, the central monitoring method comprising:
(a) transmitting, by the plurality of artificial ventilators supplying the oxygen to the plurality of patients, transmit the plurality of pieces of patient condition information to the communication unit;
(b) transmitting, by the image-capturing unit, the patient-capturing information obtained by capturing the plurality of patients in real time to the communication unit;
(c) storing, by the database unit, the plurality of pieces of patient condition information and the patient-capturing information transmitted from the communication unit;
(d) displaying, by the display unit, the plurality of pieces of patient condition information on a screen; and
(e) notifying, by the control unit, an emergency situation based on a result of comparing the plurality of pieces of patient condition information with the plurality of pieces of preset patient condition information.

8. The central monitoring method using artificial ventilators of claim 7, wherein the plurality of patients include a first patient, a second patient, a third patient, and a fourth patient who are recovering in the four bedrooms arranged inside the hospital room, respectively, and step (a) comprises:
(a1) by a first artificial ventilator among the plurality of artificial ventilators, supplying the oxygen to the first patient, and transmitting first patient condition information to the communication unit;
(a2) by a second artificial ventilator among the plurality of artificial ventilators, supplying the oxygen to the second patient, and transmitting second patient condition information to the communication unit;
(a3) by a third artificial ventilator among the plurality of artificial ventilators, supplying the oxygen to the third patient, and transmitting third patient condition information to the communication unit; and
(a4) by a fourth artificial ventilator among the plurality of artificial ventilators, supplying the oxygen to the fourth patient, and transmitting fourth patient condition information to the communication unit.

9. The central monitoring method using artificial ventilators of claim 8, wherein in step (d), the display unit displays the first to fourth patient condition information on the screen which is divided into four.

10. The central monitoring method using artificial ventilators of claim 8, wherein step (e) comprises:
(e1) receiving, by the control unit, the first to fourth patient condition information and the first to fourth preset patient condition information that were pre-stored in the database unit;
(e2) comparing, by the control unit, the first to fourth patient condition information with the first to fourth preset patient condition information, respectively, to determine whether there is the emergency patient among the first to fourth patients;
(e3) determining, by the control unit, a patient for which at least one of the first to fourth patient condition information is measured as the emergency patient, if at least one of the first to fourth patient condition information deviates from at least one of the first to fourth preset patient condition information;
(e4) generating, by the control unit, the alarm sound;
(e5) controlling, by the control unit, the display unit so that the at least one piece of patient condition information measured from the emergency patient among the plurality of pieces of patient condition information displayed on the display unit flashes in red; and
(e6) transmitting, by the control unit, the emergency signal and the patient-capturing information to the user terminal possessed by the doctor in charge.
